(19) United States
(12) Patent Application Publication
(10) Pub. No.: US 2015/0139902 A1
(43) Pub. Date: May 21, 2015

(54) PURIFICATION OF [18F] - FLUCICLATIDE

(71) Applicant: GE HEALTHCARE LIMITED, LITTLE CHALFONT, BUCKINGHAMSHIRE (GB)

(72) Inventors: Torgrim Engell, Oslo (NO); Dimitrios Mantzilas, Oslo (NO); Julian Grigg, Amersham (GB)

(73) Assignee: GE HEALTHCARE LIMITED, LITTLE CHALFONT, BUCKINGHAMSHIRE (GB)

(21) Appl. No.: 14/401,255

(22) PCT Filed: May 23, 2013
(86) PCT No.: PCT/EP2013/060588

§ 371 Date: Nov. 14, 2014

(30) Foreign Application Priority Data
May 24, 2012 (GB) 1209082.5

(51) Int. Cl. A61K 51/08; C07K 7/06

(52) U.S. Cl. CPC A61K 51/082; C07K 7/06
USPC 424/1.69; 422/159

(57) ABSTRACT

The present invention relates to a method of purification of [¹⁸F]-fluciclatide via solid phase extraction (SPE). The method is amenable to automation, and is suitable for use in conjunction with automated synthesizer apparatus—especially cassette-based synthesizers. Also provided are cassettes for carrying out the purification method.

### FIELD OF THE INVENTION

The present invention relates to a method of purification of [¹⁸F]-fluciclatide via solid phase extraction (SPE). The method is amenable to automation, and is suitable for use in conjunction with automated synthesizer apparatus—especially cassette-based synthesizers. Also provided are cassettes for carrying out the purification method.

### BACKGROUND TO THE INVENTION

Fluciclatide is the recommended INN (US Approved Name) for [¹⁸F]-AH111585. [¹⁸F]-AH111585 has been described in both patents and publications, as a PET imaging radiotracer which targets integrin receptors in vivo.

WO 03/006491 discloses compounds of Formula (I):

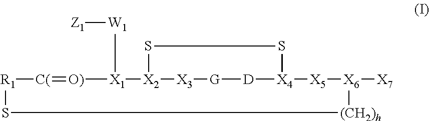

or pharmaceutically acceptable salt thereof
wherein:
G represents glycine
D represents aspartic acid
R₁ represents —(CH₂)ₙ— or —(CH₂)ₙ—C₆H₄— wherein
n represents a positive integer 1 to 10,
h represents a positive integer 1 or 2,
X₁ represents an amino acid residue wherein said amino acid possesses a functional side-chain such as an acid or amine,
X₂ and X₄ represent independently an amino acid residue capable of forming a disulfide bond,
X₃ represents arginine, N-methylarginine or an arginine mimetic,
X₅ represents a hydrophobic amino acid or derivatives thereof,
X₆ represents a thiol-containing amino acid residue,
X₇ is absent or represents a biomodifier moiety,
Z₁ represents an anti-neoplastic agent, a chelating agent or a reporter moiety and
W₁ is absent or represents a spacer moiety.

WO 2006/030291 discloses the synthesis of [¹⁸F]-fluciclatide and radiopharmaceutical compositions containing the same. WO 2006/030291 states that the radiofluorinated peptides of the invention can be prepared rapidly and efficiently, and still have the desired biological activity—of targeting integrin receptors in vivo.

Glaser et at [Bioconj.Chem., 19(4), 951-957 (2008)], disclose the synthesis and radiolabelling of [¹⁸F]-fluciclatide. Glaser et at state that the radiochemical purity was 96%, and that radio-HPLC analysis of the reaction mixture after 10 minutes incubation indicated almost quantitative coupling efficiency, with only a trace of fluorobenzaldehyde remaining Glaser et at use HPLC purification of [¹⁸F]-fluciclatide, but such methodology is recognised as being unsuitable for radiotracer automation, as well as being expensive and labour-intensive to operate.

[¹⁸F]-fluciclatide has been reported to be useful for imaging breast cancer in human patients [Kenny et al, J.Nucl.Med., 49(6), 879-886 (2008)], as well as for determining changes in tumour vascularity after anti-cancer therapy [Morrison et al, J.Nucl.Med., 50(1), 116-122 (2009)].

WO 2008/146316 discloses an SPE purification procedure for [¹⁸F]-fluorothymidine. Pascali et at [Nucl.Med.Biol., doi:10.1016/j.nucmedbio.2011.10.005 (2011)] subsequently published that the method can be used to obtain ethanol-free [¹⁸F]-fluorothymidine solutions using automated synthesizers.

Liu et at [Nucl.Med.Biol., 37, 917-925 (2010)] disclose that SPE purification can be used in the automated synthesis of [¹⁸F]-AV-45. Liu et at noted that the SPE purification did not eliminate non-polar, non-radioactive impurities.

WO 2011/044422 discloses a method of purifying [¹⁸F]-fluciclatide comprising the following steps:

(a) passing a diluted crude product reaction mixture comprising fluciclatide through a first reverse phase SPE cartridge;
(b) washing said first reverse phase SPE cartridge with a water/acetonitrile, tetrahydrofuran(THF)/water, methanol(MeOH)/water or isopropanol/water mixture; preferably, a water/acetonitrile mixture;
(c) rinsing said first reverse phase SPE cartridge with water once step (b) is completed;
(d) eluting said first reverse phase SPE cartridge with acetonitrile or tetrahydrofuran; preferably, acetonitrile;
(e) directly passing the mixture from said eluting step (d) through a normal phase SPE cartridge to give an acetonitrile or tetrahydrofuran solution; preferably, an acetonitrile solution, comprising purified fluciclatide;
(f) diluting said acetonitrile or tetrahydrofuran solution; preferably, an acetonitrile solution, comprising purified fluciclatide, with water to form a diluted water/acetonitrile or a diluted water/tetrahydrofuran solution; preferably, a diluted water/acetonitrile solution, comprising purified fluciclatide, wherein said water/acetonitrile solution contains about 40-70% (v/v) water; preferably at least about 40% (v/v) water; more preferably at least about 50% (v/v) water;
(g) passing the diluted water/acetonitrile or diluted water/tetrahydrofuran solution; preferably, diluted water/acetonitrile solution, comprising purified fluciclatide of step
(f) through a second reverse phase SPE cartridge and trapping the fluciclatide on said cartridge second reverse phase SPE cartridge;
(h) rinsing said second reverse phase SPE cartridge with water; and
(i) eluting the trapped purified fluciclatide from second reverse phase SPE cartridge with an injectable organic solvent; preferably, ethanol or DMSO; preferably with ethanol.

The method of WO 2011/044422 requires the use of a first reverse phase SPE cartridge [steps (a)-(d)]; a normal phase SPE cartridge [step (f)] and a second reverse phase SPE cartridge [steps (g) and (h)]. Each reverse phase SPE cartridge has a chain length longer than C8, preferably longer than C18, most preferably C30. Several of the solvents used in the method (e.g. THF, acetonitrile and DMSO) are not really suitable for pharmaceutical formulation. Removal and replacement of such unsuitable solvents typically requires additional steps (e.g. evaporation or solvent exchange on a cartridge), which increases process times, reduces yields and adds complexity to the process. In addition, the normal phase method of WO 2011/044422 requires that the agent is dissolved in 100% non-aqueous (i.e. organic solvents). That is expected to pose significant solubility problem for the peptides involved and risks peptide precipitation.

There is therefore still a need for an [¹⁸F]-fluciclatide purification method which is suitable for automation, in particular to give radiopharmaceutical compositions.

### THE PRESENT INVENTION

The present invention provides an [¹⁸F]-fluciclatide purification method based on SPE cartridges, which is suitable for automation. The method is much simpler than the prior art method of WO 2011/044422, since only one type of SPE column is needed. In addition, the present method employs only pharmaceutically acceptable solvents. Hence, the method is readily applied to the routine manufacture of radiopharmaceutical compositions without the need for additional processing to remove potentially toxic solvents.

The present method is effective at reducing significantly the level of radioactive and non-radioactive impurities in [¹⁸F]-fluciclatide. A particular problem is dimethylaminobenzaldehyde (DMAB; a side-product of ¹⁸F-fluorobenzaldehyde synthesis) which also reacts with the aminooxy-functionalised peptide precursor (“Precursor 1”), forming a DMAB-peptide conjugate (“Impurity A”). This DMAB-peptide conjugate is an analogue of fluciclatide, which has proven difficult to remove from [¹⁸F]-fluciclatide, since it tends to co-elute under a variety of conditions. The present method includes an acidification step, which is crucial in ensuring the removal of the DMAB-peptide conjugate impurity. The present method also removes starting aminooxy-functionalised peptide (“Precursor 1”). The present method removes 80 to 95% of peptide-related impurities, as well as aniline (which is used as a catalyst and radiostabiliser for the conjugation reaction).

### DETAILED DESCRIPTION OF THE INVENTION

In a first aspect, the present invention provides a method of purification of [¹⁸F]-fluciclatide which comprises the following steps:
(a) acidifying the [¹⁸F]-fluciclatide solution to be purified with an acidic solution, which comprises an acid having a biocompatible anion in aqueous solvent at pH 1.5 to 3.5;
(b) passing the acidified solution from step (a) through at least one C1-C4 reverse phase SPE cartridge;
(c) washing the SPE cartridge from step (b) with a first aqueous ethanol solution which comprises an acidic solution of an acid having a biocompatible anion in aqueous solvent at pH 1.5 to 3.5 and an ethanol content of 10 to 30% v/v;
(d) rinsing the washed SPE cartridge from step (c) with water or aqueous buffer solution;
(e) eluting the rinsed SPE cartridge of step (d) with a second aqueous ethanol solution having an ethanol content of 70 to 90% v/v, wherein the eluent comprises purified [¹⁸F]-fluciclatide in 70 to 90% v/v aqueous ethanol solution.

The term “[¹⁸F]-fluciclatide” refers to the compound of Formula I:

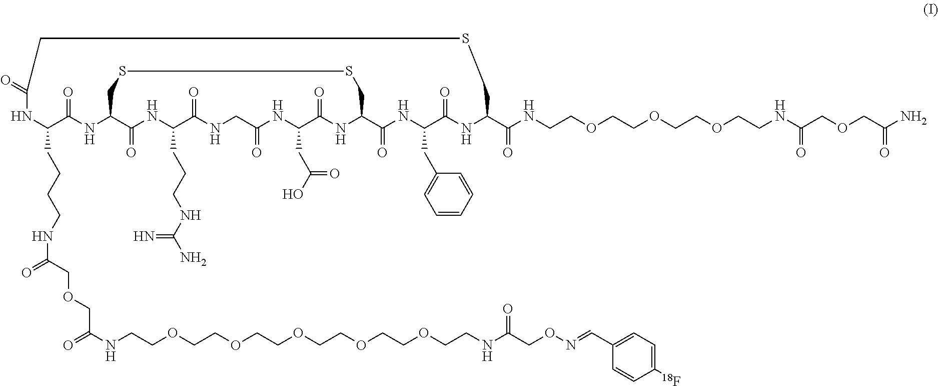

Fluciclatide (¹⁸F) is the recommended INN (US Approved Name) for [¹⁸F]-AH111585. The chemical structure of Formula (I) shows the oxime ether as the trans isomer. The term “[¹⁸F]-fluciclatide” as used herein encompasses a mixture of the cis and trans isomers, as well as substantially pure separated cis-isomer or trans-isomer.

The terms “comprises” or “comprising” have their conventional meaning throughout this application and imply that the composition must have the components listed, but that other, unspecified compounds or species may be present in addition. The term ‘comprising’ includes as a preferred subset “consisting essentially of” which means that the composition has the components listed without other compounds or species being present.

The aqueous acidic solution of steps (a) and (c) suitably has a pH in the range 1.5 to 3.5. That is to ensure that various basic impurities (including aniline pKa 4.6) are protonated. The aqueous acidic solution used in steps (a) and (c) may be the same or different, but preferably comprises the same aqueous acid. In step (a), this is used as an aqueous solution, whereas in step (c) it is used in mixture with ethanol to give the first aqueous ethanol solution.

By the term “biocompatible anion” is meant a negatively charged counterion which forms the anion of the acid, where said negatively charged counterion is non-toxic and hence suitable for administration to the mammalian body, especially the human body. The anion is suitably singly- or multiply-charged, as long as a charge-balancing amount is present. The anion is suitably derived from an inorganic or organic acid. Examples of suitable inorganic anions include: halide ions such as chloride or bromide; sulfate; nitrate; phosphate and borate. Examples of suitable organic anions include: phosphate, citrate; acetate, tartrate, lactate, pyruvate, trifluoroacetate, succinate, fumarate, maleate, methanesulfonate, ethanesulfonate, p-toluenesulfonate and benzenesulfonate.

The term “SPE cartridge” refers to a solid phase extraction cartridge. The term “solid phase extraction” has its conventional meaning. The reverse phase SPE cartridge includes a commercially available sorbent packed between two porous media layers within an elongated cartridge body. The cartridge body includes luer fittings for simplified connection. FIG. 2 provides a side elevation of a typical SPE cartridge construction. Suitable assembled reverse phase SPE cartridges for use in the present invention can be any assembled reverse phase SPE cartridge known in the art including, but not limited to, those commercially available from Waters Corporation, 34 Maple Street Milford Mass. 01757 USA. Suitable sorbents for use in a reverse phase SPE cartridge can be any sorbent known in the art including, but not limited to those, commercially available from Waters Corporation.

The term “reverse phase” has its conventional meaning in chromatography, and refers to the use of a non-polar (lipophilic) stationary phase and a polar (hydrophilic) mobile phase. Historically, most liquid chromatography used to be carried out using unmodified silica or alumina with a hydrophilic surface chemistry and a stronger affinity for polar compounds—hence it was considered “normal”. The introduction of alkyl chains bonded covalently to the support surface reversed the order of elution order. In reverse phase chromatography, polar compounds are eluted first while non-polar compounds are retained.

The term “C1-C4” refers to the number of carbon atoms in the SPE cartridge stationary phase.

In the method of the first aspect, steps (a) to (e) are suitably carried out in the alphabetical sequence shown.

Applicants believe that the acidification step (a) is an important part of the purification. Thus, when [¹⁸F]-fluorobenzaldehyde ([¹⁸F]-FBA) is prepared by conventional radiosynthesis from TMAB, up to over 95% of the chemical impurities present are derived from TMAB, DMAB and HBA:

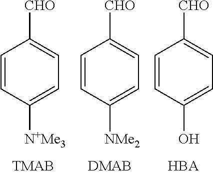

TMAB and HBA are removed via purification of the [¹⁸F]-FBA. DMAB, however, remains as an impurity (as noted above), and also reacts with Precursor 1 to form a DMAB-peptide conjugate (Impurity A). The chemical structure of Impurity A (see Table 1) shows the oxime ether as the trans isomer. The term “Impurity A” as used herein encompasses a mixture of the cis and trans isomers, as well as substantially pure separated cis-isomer or trans-isomer.

The present inventors have found that Impurity A tends to co-elute with [¹⁸F]-fluciclatide under a variety of conditions. If neutral conditions are used, Impurity A cannot be separated. Poor resolution results, and the aminooxy precursor (Precursor 1) tends to bleed through the SPE column and becomes an impurity in the [¹⁸F]-fluciclatide product. Acidification is also important to ensure that any basic species such as aniline are protonated and hydrophilic. The purification method of the present invention successfully reduces the levels of Precursor 1 and Impurity A by 90% (confirmed by mass balance control), and also removes aniline.

In step (b) of the method of the first aspect, the [¹⁸F]-fluciclatide is bound to the reverse phase SPE cartridge, and any soluble impurities remain in solution (and are discarded). The ethanol content of the first aqueous ethanol solution of washing step (c) is chosen such that the [¹⁸F]-fluciclatide has a greater affinity for the reverse phase SPE cartridge than the eluent, and consequently remains bound to the sorbent. The impurities have a weaker affinity for the stationary phase, and hence remain in the eluent, and are washed to waste. The present inventors have found that the radiochemical impurities are reduced by about 90%, and other non-radioactive impurities such as aniline are removed at this stage. The main radiochemical impurity removed is [¹⁸F]-FBA, which can be present as up to 10% in crude (i.e. unpurified) [¹⁸F]-fluciclatide. After SPE purification according to the present invention, the level of [¹⁸F]-FBA FBA is reduced to less than 1%.

The rinsing step (d), removes any residual ethanol to waste, and again, the desired [¹⁸F]-fluciclatide remains bound to the reverse phase SPE cartridge. The elution step (e) employs a second aqueous ethanol solution chosen to have a much higher ethanol content than the first aqueous ethanol solution. The [¹⁸F]-fluciclatide now has a greater affinity for the eluent than the reverse phase SPE cartridge, and consequently elutes in the second aqueous ethanol solution. The purified [¹⁸F]-fluciclatide is collected in the eluent from elution step (e).

The method of the first aspect is suitably carried out at a temperature in the range 17 to 60° C., preferably 20 to 34° C.

### Preferred Features.

In the method of the first aspect, the reverse phase SPE cartridge preferably has a carbon loading of 2 to 10%. The term “carbon loading” has its conventional meaning, and is a measure of the amount of bonded phase bound to the surface of the sorbent. The reverse phase SPE cartridge is more preferably either a C4 cartridge or a C2 cartridge, more preferably a C2 trifunctional cartridge (“tC2 cartridge”). A preferred such C2 trifunctional cartridge is an tC2 SepPak SPE column, which is commercially available from Waters Associates. The “t” of tC2 stands for trifunctional, and refers to the manufacturing, the linking of the C2 chains to the stationary phase. The tC2 SPE cartridges have a much higher carbon load and are thus much more hydrophobic than conventional C2 columns. This makes them more robust and reproducible. Most importantly, the tC2 SPE column can cope with the high peptide loading and high volumes of solution which are necessary to purify [¹⁸F]-fluciclatide.

The amount of stationary phase in the SPE cartridge determines how many cartridges are needed. For the present process, preferably around 800 mg of stationary phase is used—which may come from two 400 mg tC2 SepPak cartridges or a single cartridge of 800 mg or more.

The acidic solution of steps (a) and (c) preferably has a pH in the range 1.5 to 2.0, and is preferably chosen from 0.1% aqueous trifluoroacetic acid, 0.1-2% aqueous formic acid, 0.1-2% aqueous acetic acid or 0.1 to 5% w/w aqueous phosphoric acid. The acidic solution more preferably comprises 0.5 to 2%, most preferably 0.7 to 1.3% aqueous phosphoric acid, with 1% w/w aqueous phosphoric acid being the ideal. Phosphoric acid is preferred since phosphoric acid as its salt is already present in the [¹⁸F]-fluciclatide formulation.

In the method of the first aspect, the “first aqueous ethanol solution” is acidic and preferably has an ethanol content of approximately 20% v/v for a tC2 SepPak cartridge. A suitable range within the term ‘approximately’ is 15-25%, preferably 16-24%, more preferably 18-22%, most preferably 19-21%.

In the method of the first aspect, the “second aqueous ethanol solution” preferably has an ethanol content of approximately 80% v/v for a tC2 SepPak cartridge. A suitable range within the term ‘approximately’ is 70-90%, preferably 75-85%, more preferably 78-82%, most preferably 79-81%.

In the method of the first aspect, said reverse phase SPE cartridge is preferably pre-conditioned with one or more of ethanol, water and 0.5% aqueous phosphoric acid. More preferably, such conditioning comprises elution with ethanol (3 mL), followed by water (10 mL), and finally 0.5% aqueous H₃PO₄ (4 mL). In this way, the cartridges are activated by the organic solvent (which opens up the pores of the sorbent), and made ready to receive the peptide. Such conditioning helps ensure consistency and hence reproducible results. Another aspect is that pure ethanol helps to reduce bioburden (by acting as a bacteriocide), and helps remove any trace impurities.

The method of the first aspect preferably further comprises the steps:
(f) diluting the purified [¹⁸F]-fluciclatide solution from step (e) with a biocompatible carrier;
(g) aseptic filtration to give an [¹⁸F]-fluciclatide radiopharmaceutical composition in a form suitable for mammalian administration, having an ethanol content of 0 to 10% v/v.

Steps (a)-(g) of this preferred embodiment are suitably carried out in the sequence (a), (b), (c), (d), (e), (f) then (g).

The term “radiopharmaceutical” has its conventional meaning, and refers to a radioactive compound suitable for in vivo mammalian administration for use in diagnosis or therapy. By the phrase “in a form suitable for mammalian administration” is meant a composition which is sterile, pyrogen-free, lacks compounds which produce toxic or adverse effects, and is formulated at a biocompatible pH (approximately pH 4.0 to 10.5, preferably 6.5 to 9.5 for the agents of the present invention) and physiologically compatible osmolality. Such compositions lack particulates which could risk causing emboli in vivo, and are formulated so that precipitation does not occur on contact with biological fluids (e.g. blood). Such compositions also contain only biologically compatible excipients, and are preferably isotonic.

Preferably, the mammal is an intact mammalian body in vivo, and is more preferably a human subject. Preferably, the radiopharmaceutical can be administered to the mammalian body in a minimally invasive manner, i.e. without a substantial health risk to the mammalian subject even when carried out under professional medical expertise. Such minimally invasive administration is preferably intravenous administration into a peripheral vein of said subject, without the need for local or general anaesthetic.

The “biocompatible carrier” is a fluid, especially a liquid, in which the radiopharmaceutical can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

To be suitable for human administration, the percentage of ethanol in the [¹⁸F]-fluciclatide radiopharmaceutical composition must be less than about 10% (v/v). Preferably, the ethanol content is 7% or less, more preferably 2 to 6%, most preferably, with about 4% being the ideal. This is achieved by eluting the purified [¹⁸F]-fluciclatide in step (e) using the minimum volume of solution (ca. 1.6 mL), and dilution with aqueous buffer solution to a final volume of ca. 40 mL. The present inventors have found that the elution of step (e) is preferably carried out such that an initial volume of eluent corresponding to the dead volume of the cartridge is discarded (since it has been shown not to contain [¹⁸F]-fluciclatide). The “dead volume” is the volume of liquid that is required to fill the SPE cartridge. That procedure has the advantage of minimising the volume of 70 to 90% ethanolic solution containing the agent, so that dilution to a pharmaceutically-acceptable ethanol content can be achieved with minimal loss of radioactive concentration (RAC). For the cartridges of the present invention, that dead volume corresponds to 0.5 mL. The “dead volume” can be determined by conventional techniques—e.g. using an inert, preferably coloured molecule that does not interact with the stationary phase (e.g. because it is very much larger than the stationary phase pore size).

In order to achieve sterility, the diluted solution from step (f) is subjected to aseptic filtration. The term “aseptic filtration”, sometimes also termed ‘sterile filtration’ has its conventional meaning. Further details are provided by K. L. Williams [Microbial Contamination Control in Parenteral Manufacturing, Marcel Dekker (2004)] and M. W. Jornitz and T. H. Meltzer [Sterile Filtration; A Practical Approach, Informa Healthcare (2000)].

In dilution step (f), biocompatible carrier preferably comprises the radioprotectant 4-aminobenzoic acid, or a salt thereof with a biocompatible cation. By the term “radioprotectant” is meant a compound which inhibits degradation reactions, such as redox processes, by trapping highly-reactive free radicals, such as oxygen-containing free radicals arising from the radiolysis of water. By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred bio compatible cations are sodium and potassium, most preferably sodium.

The radioprotectant of the present invention is suitably chosen from para-aminobenzoic acid (i.e. 4-aminobenzoic acid) and salts thereof with a biocompatible cation. These radioprotectants are commercially available, including in pharmaceutical grade purity. For para-aminobenzoic acid and sodium para-aminobenzoate, a suitable concentration range is 0.5 to 4.0, preferably 1.0 to 3.0, more preferably 1.5 to 2.5, most preferably 1.8 to 2.2 mg/mL. 2.0 mg/mL is especially preferred. The radioprotectant of the present invention preferably comprises sodium para-aminobenzoate. An additional radioprotectant may also optionally be present. More preferably, the radioprotectant of the present invention consists essentially of para-aminobenzoic acid or a salt thereof with a biocompatible cation. Most preferably, the radioprotectant of the present invention consists essentially of sodium para-aminobenzoate.

Preferably, the grade of radioprotectant used is pharmaceutical grade. Thus, technical grade material has been shown to give rise to additional chemical impurities in the radiopharmaceutical composition.

When the method of the first aspect is used to provide an [¹⁸F]-fluciclatide radiopharmaceutical composition, the composition is suitably provided in a pharmaceutical grade container. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity.

The [¹⁸F]-fluciclatide radiopharmaceutical composition is suitably provided in a container wherein the headspace gas contains 5 to 30%, preferably 10-25%, most preferably 18-22% oxygen. Ideally, the headspace gas is air.

The radiopharmaceutical composition may contain additional optional excipients such as: an antimicrobial preservative, pH-adjusting agent, filler, solubiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; ethanol, phenol; cresol; cetrimide and thiomersal. Preferred antimicrobial preservative(s) are the parabens or ethanol.

The term “pH-adjusting agent” means a compound or mixture of compounds useful to ensure that the pH of the composition is within acceptable limits (approximately pH 4.0 to 10.5, preferably 6.5 to 9.5 for the agents of the present invention) for human or mammalian administration. Suitable such pH-adjusting agents include pharmaceutically acceptable buffers, such as tricine, phosphate, acetate or TRIS [i.e. tris(hydroxymethyl)aminomethane], and pharmaceutically acceptable bases such as sodium carbonate, sodium bicarbonate or mixtures thereof.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

By the term “solubiliser” is meant an additive present in the composition which increases the solubility of the radiopharmaceutical in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates (e.g. Tween™); poly(oxyethylene)poly(oxypropylene)poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

Preferred solubilisers are cyclodextrins, C₁₋₄ alcohols, polysorbates and Pluronics™, more preferably cyclodextrins and C₂₋₄ alcohols. When the solubiliser is an alcohol, it is preferably ethanol or propanol, more preferably ethanol. Ethanol has potentially a dual role, since it can also function as a biocompatible carrier and as an antimicrobial preservative. When the solubiliser is a cyclodextrin, it is preferably a gamma cyclodextrin, more preferably hydroxypropyl-β-cyclodextrin (HPCD). The concentration of cyclodextrin can be from about 0.1 to about 40 mg/ml, preferably between about 5 and about 35 mg/ml, more preferably 20 to 30 mg/ml, most preferably around 25 mg/ml.

The method of the first aspect, comprising steps (a)-(e) or (a)-(f) is preferably automated. Such automation is preferably carried out using an automated synthesizer apparatus. When such automation is used, the [¹⁸F]-fluciclatide “solution to be purified” of step (a) is preferably the crude reaction mixture direct from the automated synthesizer apparatus. When an automated synthesizer is used, the radioactive concentration (RAC) of the [¹⁸F]-fluciclatide solution at End of Synthesis (EOS) is preferably in the range 100-860, more preferably 200-700, most preferably 250-600 MBq/mL.

By the term “automated synthesizer” is meant an automated module based on the principle of unit operations as described by Satyamurthy et at [Clin.Positr.Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical composition is desired. They are commercially available from a range of suppliers [Satyamurthy et al, above], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours.

The automated synthesizer preferably comprises a cassette. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (e.g. solid phase extraction or SPE). The cassette always comprises a reaction vessel. Such reaction vessels are preferably 0.5 to 10 mL, more preferably 0.5 to 5 mL and most preferably 0.5 to 4 mL in volume and are configured such that 3 or more ports of the cassette are connected thereto, to permit transfer of reagents or solvents from various ports on the cassette. Such transfer is effected via gas pressure (typically nitrogen gas) controlled from the automated synthesizer connected to the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

The cassette is suitably disposable or single use cassette which comprises all the reagents, reaction vessels and apparatus necessary to carry out the preparation of a given batch of radio fluorinated radiopharmaceutical. The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

A preferred single use cassette for us in the method of the first aspect comprises:
(i) a vessel suitable for containing the [¹⁸F]-fluciclatide solution to be purified;
(ii) one or more C1-C4 reverse phase SPE cartridges;
(iii) a supply of the acidic solution, which comprises an acid having a biocompatible anion in aqueous solution at pH 1.5 to 3.5% as defined above;
(iv) a supply of a first aqueous ethanol solution as defined above;
(v) a supply of a second aqueous ethanol solution as defined above;
(vi) a supply of pure ethanol to condition the stationary phase.

In addition to the dilution step (f) and aseptic filtration step (g), the method of the first aspect preferably further comprises:
(h) dispensing the [¹⁸F]-fluciclatide radiopharmaceutical composition of step (f) into one or more unit dose syringes.

Steps (a)-(h) of this preferred embodiment are suitably carried out in the sequence (a), (b), (c), (d), (e), (f), (g) then (h).

The radiopharmaceutical composition of the first aspect may also be provided in a syringe. Pre-filled syringes are designed to contain a single human dosage, or “unit dose” and are therefore preferably a single-use or other syringe suitable for clinical use. The radiopharmaceutical syringe is preferably provided with a syringe shield to minimise radiation dose to the operator.

Pharmaceutical grade pABA and sodium para-aminobenzoate are commercially available, and can be obtained from e.g. Sigma or Merck. [¹⁸F]-fluciclatide and [¹⁸F]-fluciclatide compositions can be prepared by reaction of a precursor of Formula II (“Precursor 1”) with a supply of [¹⁸F]fluoride:

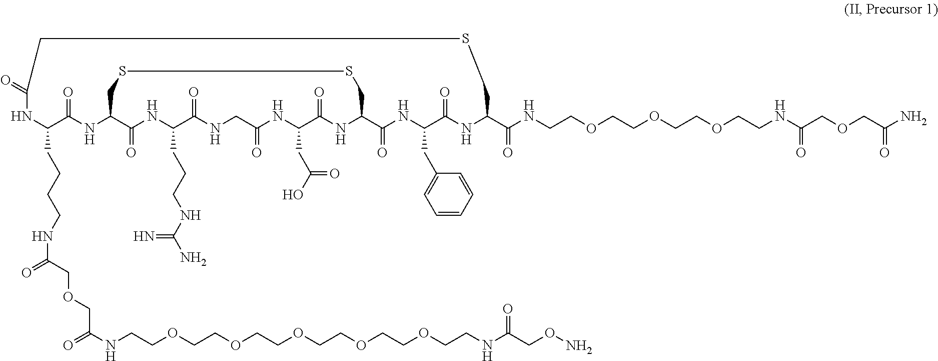

Precursor 1 is non-radioactive. It can be prepared as described by Indrevoll et at [Bioorg.Med.Chem.Lett., 16, 6190-6193 (2006)] and in the present Examples. The supply of [¹⁸F]fluoride may either be:
(i) delivered directly from a cyclotron and formulated using an ion exchange cartridge and appropriate eluent or
(ii) in the form of GMP [¹⁸F]NaF produced on an automated platform in a GMP facility.

The production of [¹⁸F]fluoride suitable for radiopharmaceutical applications is well-known in the art, and has been reviewed by Hjelstuen et at [Eur.J.Pharm.Biopharm., 78(3), 307-313 (2011)], and Jacobson et at [Curr.Top.Med.Chem., 10(11), 1048-1059 (2010)]. [¹⁸F]NaF can be produced using an “automated synthesizer” as described above.

In a second aspect, the present invention provides a cassette for carrying out the automated synthesizer method of the preferred embodiment of the first aspect, wherein said cassette comprises:
(i) a vessel suitable for containing the [¹⁸F]-fluciclatide solution to be purified;
(ii) one or more C1-C4 reverse phase SPE cartridges;
(iii) a supply of an acidic solution which comprises an acid having a biocompatible anion in aqueous solution at pH 1.5 to 3.5% 1% aqueous phosphoric acid, as defined in the first aspect;
(iv) a supply of a first aqueous ethanol solution as defined in the first aspect;
(v) a supply of a second aqueous ethanol solution as defined in the first aspect.

Preferred aspects of the reverse phase SPE cartridge, acidic solution, first aqueous ethanol solution and second aqueous ethanol solution in the second aspect, are as described in the first aspect (above).

The cassette preferably comprises the radioprotectant which is provided as a solution. The solvent for such solutions is preferably a biocompatible carrier as described above. Such solutions are preferably stored in the dark.

In a third aspect, the present invention provides the use of an automated synthesizer apparatus to carry out the automated method of the preferred embodiment of the first aspect. Preferred aspects of the method of purification and automated synthesizer in the third aspect are as described in the first aspect (above).

In a fourth aspect, the present invention provides the use of the cassette of the second aspect to carry out the automated method of the preferred embodiment of the first aspect. Preferred aspects of the method of purification and cassette in the fourth aspect are as described in the first and second aspects respectively (above).

### DESCRIPTION OF THE FIGURES

FIG. 1 shows a cassette configuration suitable for use in conjunction with a FASTlab™ automated synthesizer apparatus (GE Healthcare Limited) for carrying out the preparation and SPE purification of [¹⁸F]-fluciclatide according to the invention.

FIG. 2 depicts an SPE cartridge [210] construction suitable for use in the present invention. Cartridge [210] includes an elongate tubular body [214] defining a cylindrical cavity [216], filled with sorbent [212]. A first end [214a] of body [214] includes a transverse annular wall [218] defining an exit aperture [220] in fluid communication with cavity [216] Annular wall [218] also supports an elongate open tubular wall [222] forming a luer tip [224]. The opposing second end [214b] of body supports an end cap [226] having a cap body [228] defining an inlet aperture in fluid communication with cavity [216]. Cap body [228] includes an outer annular rim [232] engaging the outer surface [234] of tubular body [214] at second end [214b] and an inner annular wall [236] engaging the inner surface [238] of tubular body [214] at second end [214b]. Cartridge [210] also includes circular disc-shaped porous filter elements [240] and [242] spanning across cavity [216] with sorbent fill therebetween. By way of illustration, cartridge [210] is generally about 49 mm in length, about 15 mm in diameter at second end [214b], about 12.0 mm in diameter at first end [214a] and cavity [216] is about 35 mm in length.

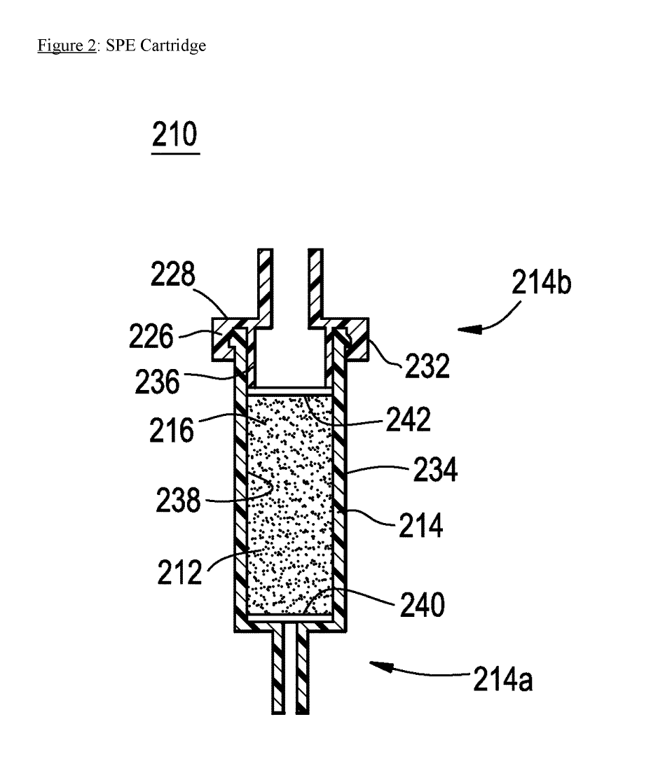

The invention is illustrated by the non-limiting Examples detailed below. Example 1 provides the synthesis of Precursor 1 of the invention. Example 2 provides the synthesis of [¹⁸F]-FBA, and Example 3 the purification of [¹⁸F]-FBA. Example 4 provides the synthesis of Compound 1 of the invention. Example 5 provides the SPE purification method of the invention.

### ABBREVIATIONS

Conventional single letter or 3-letter amino acid abbreviations are used.
Ac: Acetyl.
ACN: Acetonitrile.
Aq: aqueous.
Boc: tert-Butyloxycarbonyl.
DIPEA: N,N-diisopropylethylamine.
DMAB: 4-(dimethylamino)benzaldehyde.
DMSO: Dimethylsulfoxide.
EOS: End of synthesis.
FBA: 4-Fluorobenzaldehyde.
Fmoc: 9-Fluorenylmethoxycarbonyl.
HATU: O-(7-Azabenzotriazol-1-yl)-N,N,N′,N′-tetramethyluronium hexafluorophosphate.
HBA: 4-hydroxybenzaldehyde.
HPLC: High performance liquid chromatography.
MCX Mixed mode cation exchange cartridge
Na-pABA: sodium para-aminobenzoate.
NMM: N-methymorpholine.
NMP: 1-Methyl-2-pyrrolidinone.
PBS: Phosphate-buffered saline.
PyAOP: (7-aza-benzotriazol-1-yloxy)tripyrrolidinophosphonium hexafluorophosphate.
PyBOP: (Benzotriazol-1-yloxy)tripyrrolidinophosphonium hexafluorophosphate.
RAC: radioactive concentration.
RCP: Radiochemical purity.
RT: room temperature.
SPE: solid-phase extraction.
tBu: tert-Butyl.
TFA: Trifluoroacetic acid.
TFP: Tetrafluorophenyl.
THF: Tetrahydofuran.
TMAB: 4-(trimethylammonium)benzaldehyde.
T_{R}: retention time.

TABLE 1
Compounds of the Invention.

| Name | Structure |
|---|---|
| Peptide 1 | 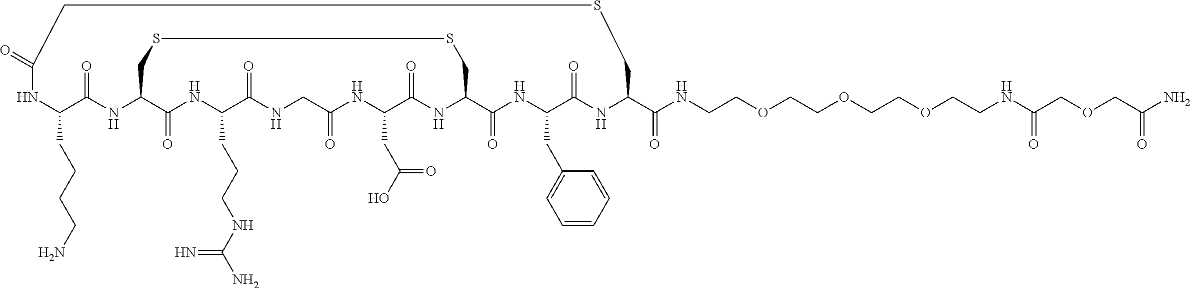 |
| Precursor 1 | 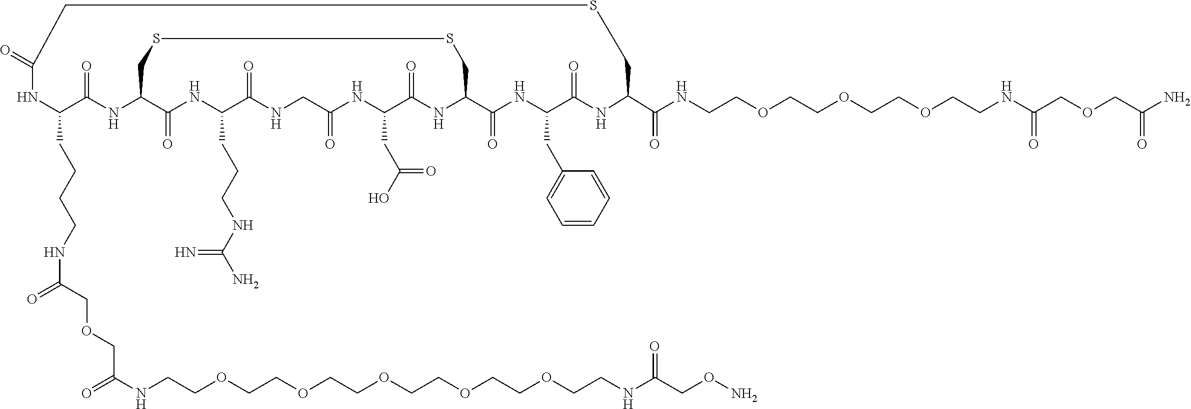 |
| Compound 1 | 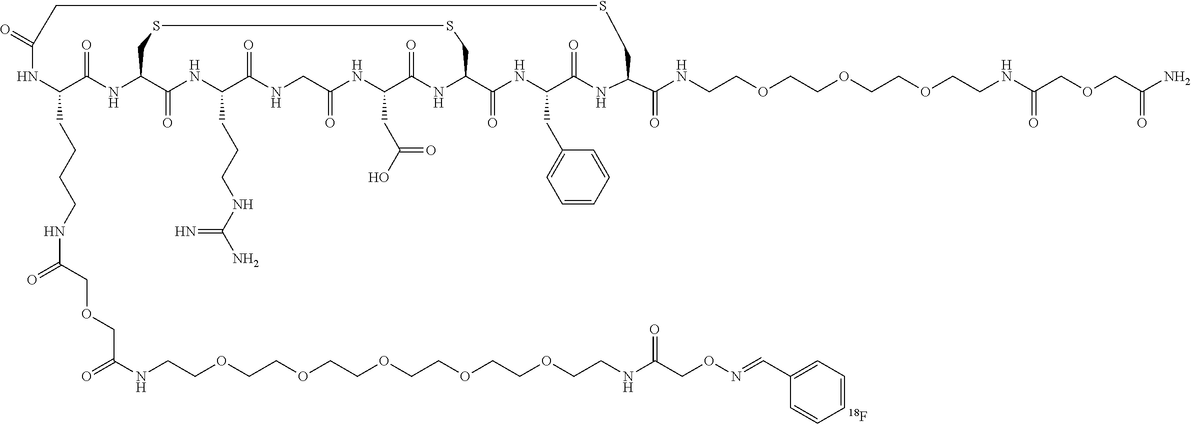 |
| Impurity A | 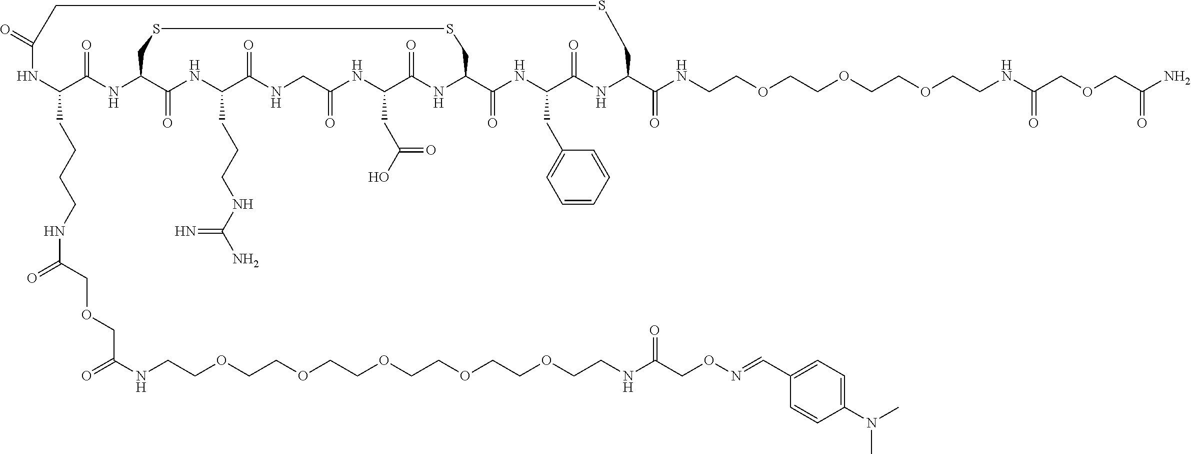 |

### Example 1

### Synthesis of Precursor 1

Peptide 1 was synthesised using standard peptide synthesis, as described by Indrevoll et at [Bioorg.Med.Chem.Lett., 16, 6190-6193 (2006)].

### (a) 1,17-Diazido-3,6,9,12,15-pentaoxaheptadecane

A solution of dry hexaethylene glycol (25 g, 88 mmol) and methanesulfonyl chloride (22.3 g, 195 mmol) in dry THF (125 mL) was kept under argon and cooled to 0° C. in an ice/water bath. A solution of triethylamine (19.7 g, 195 mmol) in dry THF (25 mL) was added dropwise over 45 min. After 1 hr the cooling bath was removed and the reaction was stirred for another for 4 hrs. Water (55 mL) was then added to the mixture, followed by sodium hydrogencarbonate (5.3 g, to pH 8) and sodium azide (12.7 g, 195 mmol). THF was removed by distillation and the aqueous solution was refluxed for 24 h (two layers were formed). The mixture was cooled, ether (100 mL) was added and the aqueous phase was saturated with sodium chloride. The phases were separated and the aqueous phase was extracted with ether (4×50 mL). The combined organic phases were washed with brine (2×50 mL) and dried (MgSO₄). Filtration and evaporation of the solvent gave a yellow oil 26 g (89%). The product was used in the next step without further purification.

### (b) 17-Azido-3,6,9,12,15-pentaoxaheptadecanamine

To a vigorously stirred suspension of 1,17-diazido-3,6,9,12,15-pentaoxaheptadecane (25 g, 75 mmol) in 5% HCl (200 mL) was added a solution of triphenylphosphine (19.2 g, 73 mmol) in ether (150 mL) over 3 hrs at room temperature. The reaction mixture was stirred for additional 24 hrs. The phases were separated and the aqueous phase was extracted with dichloromethane (3×40 mL). The aqueous phase was cooled in an ice/water bath and the pH was adjusted to 12 by addition of solid potassium hydroxide. The aqueous phase was concentrated and the product was taken up in dichloromethane (150 mL). The organic phase was dried (Na₂SO₄) and concentrated giving a yellow oil 22 g (95%). The product was identified by electrospray mass spectrometry (ESI-MS) (MH+ calculated: 307.19; found 307.4). The crude oil was used in the next step without further purification.

### (c) 23-Azido-5-oxo-6-aza-3,9,12,15,18,21-hexaoxatricosanoic acid

To a solution of 17-azido-3,6,9,12,15-pentaoxaheptadecanamine (15 g, 50 mmol) in dichloromethane (100 mL) was added diglycolic anhydride (Acros, 6.4 g, 55 mmol). The reaction mixture was stirred overnight. The reaction was monitored by ESI-MS analysis, and more reagents were added to drive the reaction to completion. The solution was concentrated to give a yellow residue which was dissolved in water (250 mL). The product was isolated from the aqueous phase by continuous extraction with dichloromethane overnight. Drying and evaporation of the solvent gave a yield of 18 g (85%). The product was characterized by ESI-MS analysis (MH+ calculated: 423.20; found 423.4). The product was used in the next step without further purification.

### (d) 23-Amino-5-oxo-6-aza-3,9,12,15,18,21-hexaoxatricosanoic acid

23-Azido-5-oxo-6-aza-3,9,12,15,18,21-hexaoxatricosanoic acid (9.0 g, 21 mmol) was dissolved in water (50 mL) and reduced using H₂(g)-Pd/C (10%). The reaction was run until ESI-MS analysis showed complete conversion to the desired product (MH+ calculated: 397.2; found 397.6). The crude product was used in the next step without further purification.

### (e) (Boc-aminooxy)acetyl-PEG(6)-diglycolic acid

A solution of dicyclohexycarbodiimide (515 mg, 2.50 mmol) in dioxan (2.5 mL) was added dropwise to a solution of (Boc-aminooxy)acetic acid (477 mg, 2.50 mmol) and N-hydroxysuccinimide (287 mg, 2.50 mmol) in dioxan (2.5 mL). The reaction was stirred at RT for 1 h and filtered. The filtrate was transferred to a reaction vessel containing a solution of 23-amino-5-oxo-6-aza-3,9,12,15,18,21-hexaoxatricosanoic acid (1.0 g, 2.5 mmol) and NMM (278 μl, 2.50 mmol) in water (5 mL). The mixture was stirred at RT for 30 min. ESI-MS analysis showed complete conversion to the desired product (MH+ calculated: 570.28; found 570.6). The crude product was purified by preparative HPLC (column: Phenomenex Luna 5μ C18 (2) 250×21.20 mm, detection: 214 nm, gradient: 0-50% B over 60 min where A=H₂O/0.1% TFA and B=acetonitrile/0.1% TFA, flow rate: 10 mL/min) affording 500 mg (38%) of pure product. The product was analyzed by HPLC (column: Phenomenex Luna 3μ C18 (2), 50×2.00 mm, detection: 214 nm, gradient: 0-50% B over 10 min where A=H₂O/0.1% TFA and B=acetonitrile/0.1% TFA, flow rate: 0.75 mL/min, Rt=5.52 min). Further confirmation was carried out by NMR analysis.

### (f) Conjugation of (Boc-aminooxy)acetyl-PEG(6)-diglycolic acid to Peptide 1

(Boc-aminooxy)acetyl-PEG(6)-diglycolic acid (0.15 mmol, 85 mg) and PyAOP (0.13 mmol, 68 mg) were dissolved in DMF (2 mL). NMM (0.20 mmol, 20 μL) was added and the mixture was stirred for 10 min. A solution of Peptide 1 (0.100 mmol, 126 mg) and NMM (0.20 mmol, 20 μL) in DMF (4 mL) was added and the reaction mixture was stirred for 25 min. Additional NMM (0.20 mmol, 20 μL) was added and the mixture was stirred for another 15 min. DMF was evaporated in vacuo and the product was taken up in 10% acetonitrile-water and purified by preparative HPLC (column: Phenomenex Luna 5μ C18 (2) 250×21.20 mm, detection: UV 214 nm, gradient: 5-50% B over 40 min where A=H₂O/0.1% TFA and B=acetonitrile/0.1% TFA, flow rate: 10 mL/min,) affording 100 mg semi-pure product. A second purification step where TFA was replaced by HCOOH (gradient: 0-30% B, otherwise same conditions as above) afforded 89 mg (50%). The product was analysed by HPLC (column: Phenomenex Luna 3μ C18 (2) 50×2 mm, detection: UV 214 nm, gradient: 0-30% B over 10 min where A=H₂O/0.1% HCOOH and B=acetonitrile/0.1% HCOOH, flow rate: 0.3 mL/min, Rt: 10.21 min). Further product characterisation was carried out using ESI-MS (MH22+ calculated: 905.4, found: 906.0).

### (g) Deprotection

Deprotection was carried out by addition of TFA containing 5% water to 10 mg of peptide.

### Example 2

### Radiosynthesis of [¹⁸F]-Fluorobenzaldehyde (¹⁸F-FBA)

[¹⁸F]-fluoride was produced using a GEMS PETtrace cyclotron with a silver target via the [¹⁸O](p,n) [¹⁸F] nuclear reaction. Total target volumes of 1.5-3.5 mL were used. The radio fluoride was trapped on a Waters QMA cartridge (pre-conditioned with carbonate), and the fluoride is eluted with a solution of Kryptofix_{2.2.2.} (4 mg, 10.7 μM) and potassium carbonate (0.56 mg, 4.1 μM) in water (80 μL) and acetonitrile (320 μL). Nitrogen was used to drive the solution off the QMA cartridge to the reaction vessel. The [¹⁸F]-fluoride was dried for 9 minutes at 120° C. under a steady stream of nitrogen and vacuum. Trimethylammonium benzaldehyde triflate, [Haka et al, J.Lab.Comp.Radiopharm., 27, 823-833 (1989)] (3.3 mg, 10.5 μM), in DMSO (1.1 mL) was added to the dried [¹⁸F]-fluoride, and the mixture heated at 105° C. for 7 minutes to produce 4-[¹⁸F]-fluorobenzaldehyde.

### Example 3

### Purification of [¹⁸F]-Fluorobenzaldehyde (¹⁸F-FBA)

The crude labelling mixture from Example 2 was diluted with ammonium hydroxide solution and loaded onto an MCX+SPE cartridge (pre-conditioned with water as part of the FASTlab sequence). The cartridge was washed with water, dried with nitrogen gas before elution of 4-[¹⁸F]-fluorobenzaldehyde back to the reaction vessel in ethanol (1.8 mL). A total volume of ethanol of 2.2 mL was used for the elution but the initial portion (0.4 mL) was discarded as this did not contain [¹⁸F]-FBA. 4-7% (decay corrected) of the [¹⁸F] radioactivity remained trapped on the cartridge.

### Example 4

### Preparation of [¹⁸F]-fluciclatide (Compound 1)

The conjugation of [¹⁸F]-FBA with Precursor 1 (5 mg) was performed in a solution of ethanol (1.8 mL) and water (1.8 mL) in the presence of aniline hydrochloride. The reaction mixture was maintained at 60° C. for 5 minutes.

### Example 5

### SPE Purification of [¹⁸F]-fluciclatide (Compound 1)

The [¹⁸F]-fluciclatide solution to be purified (from Example 4) was purified as follows:

### (a) Conditioning

Two 400 mg tC2 SPE cartridges (Waters Associates) were conditioned using (i) ethanol (3 mL), (ii) water (10 mL), then 0.5% H₃PO₄ (aq) (4 mL).

### (b) Loading.

The [¹⁸F]-fluciclatide solution to be purified (from Example 4) was diluted 1:1 with 1% H₃PO₄ (aq) (4 mL) and transferred to the conditioned SPE cartridge of step (b) via syringe. The reaction vessel containing the unpurified [¹⁸F]-fluciclatide was rinsed with 1% H₃PO₄ (aq) (1 mL) then water (3 mL), and the washings also transferred though the conditioned SPE cartridge.

### (c) Purification.

The loaded cartridge from step (c) was washed with 20.1% EtOH/79.9% 1% H₃PO₄ (aq) (2×5.75 mL). The SPE column was then washed with water (2 mL), and flushed with nitrogen gas.

### (d) Elution.

The purified [¹⁸F]-fluciclatide was eluted from the SPE cartridge of step (c) using 80% EtOH/20% water (1.5 mL), followed by flushing with nitrogen gas. The eluted [¹⁸F]-fluciclatide was transferred to a vial for pharmaceutical formulation and dispensing.

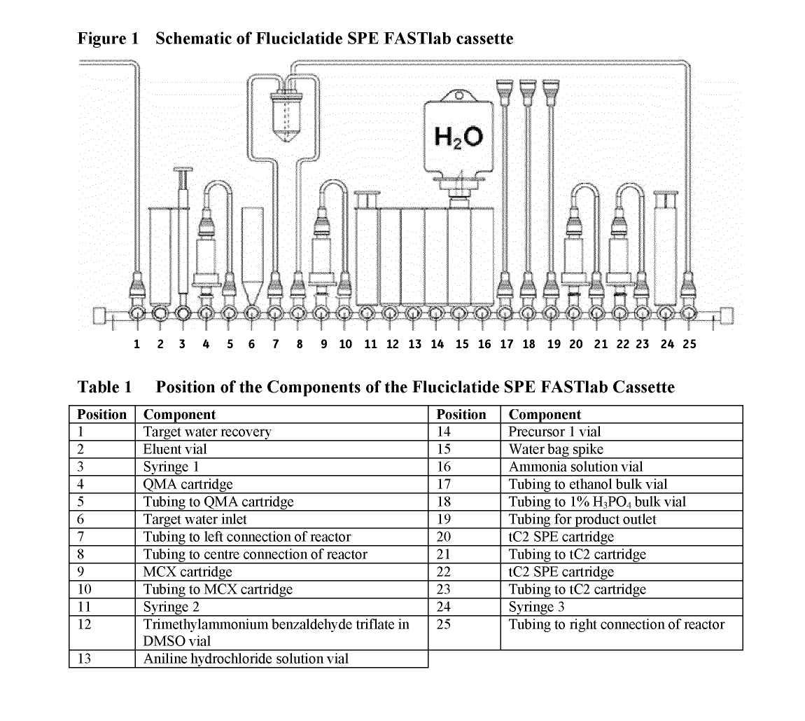

A cassette configuration suitable for use in conjunction with a FASTlab™ automated synthesizer apparatus (GE Healthcare Limited) for carrying out the preparation and SPE purification of [¹⁸F]-fluciclatide according to the invention is shown in FIG. 1.

What is claimed is:

1. A method of purification of [¹⁸F]-fluciclatide which comprises the following steps:
(a) acidifying the [¹⁸F]-fluciclatide solution to be purified with an acidic solution, which comprises an acid having a biocompatible anion in aqueous solvent at pH 1.5 to 3.5;
(b) passing the acidified solution from step (a) through at least one C1-C4 reverse phase SPE cartridge;
(c) washing the SPE cartridge from step (b) with a first aqueous ethanol solution which comprises an acidic solution of an acid having a biocompatible anion in aqueous solvent at pH 1.5 to 3.5 and an ethanol content of 10 to 30% v/v;
(d) rinsing the washed SPE cartridge from step (c) with water or aqueous buffer solution;
(e) eluting the rinsed SPE cartridge of step (d) with a second aqueous ethanol solution having an ethanol content of 70 to 90% v/v, wherein the eluent comprises purified [¹⁸F]-fluciclatide in 70 to 90% v/v aqueous ethanol solution.

2. The method of claim 1, wherein the acidic solution of steps (a) and (c) each independently comprise 0.1% trifluoroacetic acid, 0.1-2% formic acid, 0.1-2% acetic acid or 0.1 to 5% phosphoric acid.

3. The method of claim 2, wherein the acidic solution of steps (a) and (c) each independently comprise 0.5 to 2% phosphoric acid.

4. The method of claim 1, wherein the reverse phase SPE cartridge has a carbon load of 2 to 10%.

5. The method of claim 1, wherein said first aqueous ethanol solution has an ethanol content of approximately 20% v/v.

6. The method of claim 1, wherein said second aqueous ethanol solution has an ethanol content of approximately 80% v/v.

7. The method of claim 1 wherein said reverse phase SPE cartridge is pre-conditioned with one or more of ethanol, water and 0.5% aqueous phosphoric acid.

8. The method of claim 1, which further comprises:
(f) diluting the purified [¹⁸F]-fluciclatide solution from step (e) with a biocompatible carrier;
(g) aseptic filtration of the diluted solution from step (f) to give an [¹⁸F]-fluciclatide radiopharmaceutical composition in a form suitable for mammalian administration, having an ethanol content of 0 to 10% v/v.

9. The method of claim 8, wherein the biocompatible carrier of step (f) comprises the radioprotectant 4-aminobenzoic acid, or a salt thereof with a biocompatible cation.

10. The method of claim 1, wherein said steps (a)-(e) or (a)-(g) are automated.

11. The method of claim 10, where the automation is carried out using an automated synthesizer apparatus.

12. The method of claim 11, where said automated synthesizer apparatus comprises a single use cassette.

13. The method of claim 12, where the single use cassette comprises:
(i) a vessel suitable for containing the [¹⁸F]-fluciclatide solution to be purified;
(ii) one or more C1-C4 reverse phase SPE cartridges;
(iii) a supply of the acidic solution as defined in any one of claims 1 to 3;
(iv) a supply of a first aqueous ethanol solution as defined in claim 1 or claim 5;
(v) a supply of a second aqueous ethanol solution as defined in claim 1 or claim 6.

14. The method of claim 8, which further comprises:
(h) dispensing the [¹⁸F]-fluciclatide radiopharmaceutical composition of step (g) into one or more unit dose syringes.

15. A cassette for carrying out the automated method according to claim 12, wherein said cassette comprises:
(i) a vessel suitable for containing the [¹⁸F]-fluciclatide solution to be purified;
(ii) one or more C1-C4 reverse phase SPE cartridges;
(iii) a supply of the acidic solution which comprises an acid having a biocompatible anion in aqueous solvent at pH 1.5 to 3.5;
(iv) a supply of a first aqueous ethanol solution which comprises an acidic solution of an acid having a biocompatible anion in aqueous solvent at pH 1.5 to 3.5 and an ethanol content of 10 to 30% v/v;
(v) a supply of a second aqueous ethanol solution which comprises an acidic solution of an acid having a biocompatible anion in aqueous solvent at pH 1.5 to 3.5 and an ethanol content of 70 to 90% v/v.

16-17. (canceled)
